(19) United States
(12) Patent Application Publication
(10) Pub. No.: US 2006/0134758 A1
(43) Pub. Date: Jun. 22, 2006

(54) Process for obtaining a ccellulosic wet sheet and a membrane, the equipment used to obtain the membrane and the membrane obtained

(76) Inventors: Nelson Luiz Ferreira Levy, Sao Paulo (BR); Edna Cristina Kurokawa, Sao Paulo (BR); Pablo Angel Sanchez Podlech, Joinville (BR)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 10/538,131

(22) PCT Filed: Nov. 26, 2003
(86) PCT No.: PCT/BR03/00174

§ 371 Date: Jan. 30, 2006

(30) Foreign Application Priority Data
Dec. 5, 2002 (BR) P102054990

(51) Int. Cl. C12P 19/04

(52) U.S. Cl. 435/101

(57) ABSTRACT

Said process used to obtain, in industrial scale, wet sheets and membranes of bacterial cellulose of high purity and specific physical and chemical properties, using the appropriate culture medium that is prepared in a mechanical mixer furnished with a jacket for temperature control and heat exchange, using selected recycled or non-recycled inoculums, fermentation in covered trays with the temperature controlled by an external heating/cooling jacket through the circulation of hot/cold water, not requiring control of humidity and air renovation, collecting of the wet sheets that are then submitted to a process of purification and whitening by successive whirlpool washing and rinsing cycles using diverse heated or non-heated aqueous solutions, being afterwards forwarded to draining and drying/dehydration, passing through a semi-continuous system of rollers and belt conveyors made of absorbent material. This invention also refers to the process used to obtain compounded cellulosic wet sheets. When a wet sheet reaches a certain thickness, screens or other artifacts of diverse materials are added to the surface of the wet sheet already pre-formed so that in a second fermentation stage such screens and artifacts are covered by the cellulosic wet sheet, thus forming a compound product.

This invention refers to a process used to obtain, In industrial scale, wet sheets and membranes of bacterial cellulose of high purity and specific physical and chemical properties. The invention also refers to the covered trays used in the fermentation; to the equipment used to obtain the membrane from the wet sheet; to the cellulosic membrane obtained through the above process, characterized by its permeability to gases and its impermeability to liquids, being therefore ideal for medical use in “in vivo ” tissue regeneration, and last but not least, to the various applications of the wet sheet and of the membrane.

### BACKGROUND OF THE INVENTION

The current state of the technique referring to the processes used to obtain cellulose through bacteriological means may be discussed in the following lines:

1) Process to Obtain the Cellulosic Wet Sheet and Culture Medium

First references made to cellulose produced by bacteria go back to 1886, with a paper by Brown, A. J. in Journal of Chemical Society. Studies on culture media for the production of cellulose were published by Tarr and Hibert in 1931; Khouvine in 1936; and Hestrin, Aschener and Mager in 1947. Khausal and Walker published in 1951 a paper also describing differentiated culture media for the production of cellulose.

In 1977, Clovin published a paper describing the effects of adding glucose to the culture medium for the production of cellulose. He also mentions the formation of a wet sheet made of cellulose fibrils that became visible after a period of 6-8 hours from the inoculation of the culture medium by the microorganisms. In a paper published in 1980, Clovin reports the extrusion of the cellulose through the bacteria's cellular wall, affirming that the microfibrils spontaneously assemble in the culture medium to form cellulose fibril. A study published in 1975 by Lepard et al. reports the emerging microfibrils as being linearly extended polyglucosan structures, at first highly hydrated, and as wide as 100 nm.

Later, still suspended in the liquid medium, they gradually assemble to form a consolidated fibril. According to the literature and to observation, these cellulose fibrils aggregate at random, resulting in a cellulosic zooglea or wet sheet that floats in the culture medium.

Bacterial cellulose is being produced for decades in Far East countries (as the Philippines and Thailand) for the production of “Nata de Coco” (coconut gel), a sweet obtained by cooking the cellulosic wet sheet in sugar syrup. In the process they use, the culture medium is coconut water or coconut milk, and it takes weeks until the fermentation is completed. Furthermore, it is a public domain domestic process where temperature and humidity are not controlled, so the quality of the product varies accordingly. As a result, it is difficult to implement an economically viable industrial operation where the quality of the product can be assured, due to the lack of regularity in the conditions and period of fermentation of the raw material.

Patent EP 83307636 (priority U.S. Pat. No. 450,324, of Dec. 16, 1982) describes the preparation of a bacterial cellulose wet sheet obtained by cultures of Acetobacter Xylinum and processed in order to substitute the culture medium in the sheet for water or other physiologically compatible liquid, to prevent the membrane from adhering to the surface of the injury. The non adherence obtained by the inclusion of physiologically compatible liquids in the object of the above mentioned patent document differs fundamentally from the basic concept of the invention presented here, since the final product of the present invention is dehydrated, creating a condition of natural adherence of the membrane to the raw bed of a wound, allowing the body to establish a microenvironment on the surface of the lesion, ideal for tissue regeneration. Contrary to the concept claimed in the European patent above, which foresees the periodical change of wet sheet dressings, the material in the present invention, in most cases, is intended for one application reducing the time and cost of patient treatment. Moreover, there is superior economic advantage in the industrial manufacture process of the wet sheet dressing since the purification and dehydration process is substantially simple and less onerous than the liquid substitution process.

Patent BR PI 9204232 describes the environmental conditions needed to obtain cellulosic wet sheets, requiring an oxygen reposition at 5 m³/h for a zooglea surface equal to 1 m². This technology presents the inconvenience of high operating costs (with the constant need to replace absolute air filters, for instance) and the need to establish strict and complex controls for several parameters (control and correction of the air humidity, for example), generating high production costs. In addition, it mentions the formation of a lamella adhered to the underneath of each zooglea, with distinctive characteristics, which must be removed.

Patents BR 8404937 of 1984 and U.S. Pat. No. 4,912,049 of 1990 describe a culture of Acetobacter xylinum where the source of nitrogen is a Tea sinensis extract and the source of carbohydrates is saccharose. Presently it is known that Acetobacter xylinum uses glucose as source of carbon. The use of saccharose by said patent reduces its reproduction speed, increasing the period of fermentation for the production of the wet sheet, since the saccharose must be broken into glucose and fructose.

Borzani and Souza (in a paper published in the World Journal of Microbiology and Biotechnology, vol. 14, 1998) demonstrated that the cellulosic wet sheet is formed in its interface with the air and not in its interface with the culture medium.

Based on this fact, our invention refers to a process used to obtain compound wet sheets, aiming at adding other physical characteristics to the final product, such as increased mechanical resistance, malleability, plasticity, thermal and/or electric conductivity and internal structural support in order to enhance the possibilities of conformation of the material at a later time.

2) Use of Cellulosic Wet Sheets

Hydrated cellulose wet sheets, when triturated, may be used as stabilizers and thickeners in milk, fruit juices and food in general, replacing the chemical stabilizers and thickeners that are generally used. It presents the advantage of being a natural product, without chemical components, in addition to being a source of dietary fibers, since it is made solely of cellulose.

Once obtained through the required process, these cellulosic wet sheets can be dried in the equipment proposed in this invention in order to obtain cellulosic membranes that are extremely fine and possess characteristic features as permeability to gases and impermeability to liquids, being ideal for medical use in “in vivo” tissue regeneration.

According to this invention, in order to aggregate properties to its features, the cellulosic wet sheet may form a compound material and go through a drying process so as to obtain products that demand such specific properties as high resistance to traction or ripping as offered by the membrane thus obtained.

3) Production of the Cellulosic Membrane

Patent BR PI 9204232 maintains that in order to obtain cellulosic wet sheets it is necessary to go through a phase of slow freezing, which in addition to generating high costs, propitiates the formation of large ice crystals that damage the microfibrilar structure of the cellulose. According to the author, this process results in a product that is impermeable to liquids, gases and solid particles, rendering this membrane inadequate to cover wounds, since its very features, specially impermeability to gases, would not allow the formation and maintenance of an environment ideal for tissue regeneration.

Patent EP 206.830.A2 refers to membranes obtained from bacterial cellulose zoogleas where the product obtained is not submitted to a dehydration process but to a controlled pressing, aiming at obtaining lower density regions through the reorientation of fibers within the membrane. The product thus obtained would be an absorbent with retained liquids preventing it from adhering to the bleeding bed of the wounds. In comparison to our invention, this product does not present a material advantage over the treatments presently in use, since such dressings, due to their non-adherence to wounds, would demand frequent handling of the patient and the resulting exposure to the danger of infections and increased costs.

Patents BR PI 8404937 of 1984 and U.S. Pat. No. 4,912,049 of 1990 describe how membranes can be obtained by drying the wet sheets stretched on stenters. In addition to this procedure being labor-intensive, the membrane would become permeable to gases and liquids when used as an artificial skin graft in cutaneous lesions.

A membrane that is permeable to liquids would not meet the requirements to preserve the medium that is ideal in promoting tissue regeneration, because, while permitting the passage of liquids it also allows the passage of electrolytes, vitamins and elements as sodium, potassium, calcium ions, vitamin K, peptides, as well as soluble fibrinogen, enzymes such as pro-thrombin and thrombin that dissolved in the exudate act as mediators involved in the coagulation cascade and in the process of tissue regeneration.

4) Use of Cellulosic Membranes

Impermeability to liquids and permeability to gases is fundamental to preserve the natural environment in the regeneration of the tissues of living organisms. In accordance with this concept, the cellulosic membrane obtained through the process of our invention presents the ideal features to promote the natural regeneration of the tissues.

Treating tissue lesions has always posed a great challenge to the medical science. Skin lesions of diverse etiologies represent a problem of extreme gravity throughout the world. As life expectancy of the world's population increases, this problem becomes more evident for the professionals in the medical area. In the last twenty years, several product lines were developed in an attempt to minimize or at least to better address the issue. Presently, the treatment of wounds focuses mainly on handling lesions resulting from traumas, pressure ulcers in elderly patients, diabetic ulcers, venous and arterial ulcers, and lesions in burned patients.

Traditionally, dressings are used to protect the wound from a potentially contaminated environment so as to allow the body to recover with a minimum interference from external agents. Gauze is still the dressing that is most widely used for this purpose.

Presently the medical science acknowledges the importance of several specialized dressings that aim at facilitating the handling and cure of this kind of wound. Cost contention is also an important factor in the development of specialized dressings that may reduce the need for frequent handling, accelerate healing, and decrease the length of hospital stay for patients with wounds.

Presently there are in the marketplace several products that may be classified in different categories:
Conventional dressings: gauze, antiseptic materials and adhesive tape to keep them in place.
Synthetic dressings: Mainly expanded polytetrafluorethylen films with adhesives.
Biological dressings: Collagen, alginate, keratin or cellulose films, as well as pig and human skin, either from cadavers or living donators.
Compound dressings: they combine materials such as plastic films with collagen, hydrocollolds, alginates, layers of activated charcoal, and other absorbent materials that present some degree of compatibility.

Such diversity of materials with alleged healing qualifies within conceptions so different from each other create a situation where medical teams are intensely involved in studies and tests of new concepts and products, but nothing has arisen that could really be considered a true revolution in skin wounds treatment.

Recent scientific literature in the area of skin lesions advocates the need for a specific dressing for each different phase of the skin regeneration process, and paradoxically hospitals everywhere in the world use gauze, cotton balls and adhesive tapes in over 90% of the procedures involved in treating skin wounds of different etiologies.

In terms of treatment of skin wounds, the most recent studies indicate a trend related to the application of bio-engineered products, specially “in vitro” skin cultures. Along these lines, one can highlight three products:

Dermagraft®, produced by Advanced Tissue Sciences, is produced through “in vitro” culture of human dermal cell (fibroblasts) upon a biosynthetic material composed of an ultra thin semi-permeable membrane, bonded to a nylon structure. This structure provides support for the growth of fibroblasts, forming a synthetic skin for temporary use; Dermagraft® is indicated for third degree burns (where the epidermis, entire dermis and underlying tissue are damaged) and replaces the treatment used today, when bums are covered with cadaver skin to prepare the wound bed for eventual placement of autograft tissue. The main disadvantage mentioned by health professionals is the high cost of the product. A 30 sq. cm piece costs US $ 3,600.00, while a cadaver skin of the same size costs from US$ 600.00 to US$ 800.00.

LifeCell, Inc.'s Alloderm® is a tissue obtained from cadaver skin. The company performs tests to determine the viability of the donation, including the medical and social history of the donor, physical exams, serology, microbiology and “causa mortis”, as well as tests performed on skin samples to ensure that there are no pathogenic fungi and bacteria. Certified labs analyze blood samples from the donor, for Hepatitis B; Hepatitis C; types 1 and 2 HIV; HTLV-1; and Syphilis. When the skin has passed successfully all these tests, its dermal and epidermal cells are removed to prevent reaction from the patient's immune system. The final result of this process is the protein structure of the basal membrane, which is then reconstructed to facilitate adherence and the epithelial migration in the wound area. Having applied the product, there is the need to place autograft tissue on the Alloderm®. Information on the market price of the product was not available, but through the description of the procedures used in its making we can infer that it is extremely costly, and it is not covered by health insurance plans in the countries it is marketed.

Organogenisis' Appligraf® is a two-layered live artificial skin. The epidermal layer is made of human keratinocytes, and the dermal layer is made of human fibroblasts on a type 1 bovine collagen support Appligraf® contains matrix and cytokine cells found in human skin but it does not contain melanocytes, macrophages, lymphocytes, blood vessels or hair follicies. This product is manufactured under aseptic conditions using human foreskin taken from newborn children. Fibroblasts and keratinocytes, that are Appligraf® sources of cells, are exhaustively tested to ensure their viability and the absence of viruses, bacterial infections, fungus, iso-enzymes and potentiality for developing tumors. The products of animal origin are also submitted to the tests mentioned above, and countries that are free of Bovine Spongiform Encephalopathy supply the bovine products.

Appligraf® is indicated for the treatment of non contaminated ulcers in patients that are either diabetic or suffer from venous insufficiency, provided that there is no exposure of muscles, tendons, capsules or bones. Appligraf® should not be used in contaminated wounds, or in patients that are allergic to bovine collagen or present hyper sensibility to agarose, medium in which Appligraf® is packed. Its selling price is approximately US$ 975.00 for a 7.5 sq. cm piece.

In comparison with the products described herein, the invention's membrane stands out for its distinguished features in terms of performance and cost, being the choice product for patients with wounds of whatever etiology, in which it provides the best treatment possible at the lowest cost. Due to its malleability and hygroscopicity, this cellulosic membrane when put on the lesion at first conforms to the wound topography without interfering in the tissue regeneration biochemistry sequence in process; the fibrinogen structures in the wound exudation are transformed in fibrin that finds anchorage spots on the membrane, making it adhere to the wound; from then on, the membrane transforms this bleeding bed in a true “in vivo” tissue culture environment, propitiating the regeneration of the wounded or absent structures, in an environment and culture medium provided by the interaction of the cellulose membrane with the regeneration processes of the person's body.

Due to its physical-chemical features, the membrane transforms a contamination-prone open wound in a protected environment that functions as an “in vivo” tissue culture, and it stays in place until the tissue is reconstructed, being thereafter eliminated naturally.

### BRIEF SUMMARY OF THE INVENTION

This invention refers to a new process to obtain bacterial cellulose wet sheets of high purity and specific physical-chemical properties in industrial scale, using the appropriate culture medium, prepared in a mechanical mixer furnished with a jacket for temperature control and heat exchange, using selected recycled or non-recycled inoculums, fermentation in covered trays with the temperature controlled by an external heating/cooling jacket through the circulation of hot/cold water (these trays are also part of this application for patent), no humidity and air renovation control required, collecting of the wet sheets that are then submitted to a process of purification and whitening characterized by successive whirlpool washing and rinsing cycles in diverse heated or non-heated aqueous solutions, being afterwards forwarded to draining and drying/dehydration passing through a semi-continuous system of rollers and belt conveyors made of absorbent material, also object of this application.

This invention also refers to the process used to obtain compounded cellulosic wet sheets. In this case, the fermentation occurs in two different stages. The first fermentation stage follows the lines described above. When the wet sheet reaches a certain thickness, screens or other artifacts of diverse materials are added to the surface of the wet sheet already pre-formed so that in a second fermentation stage such screens and artifacts are covered by the cellulosic wet sheet, thus forming a compound product. When the fermentation is completed, the compound wet sheet goes through the purifying and whitening procedures and the process goes on as described above.

### BRIEF DESCRIPTION OF THE DRAWINGS

In order to ensure a better understanding of this invention, we have annexed figures that represent in an outlined though not limiting manner the following:

FIG. 1—Flow chart of the process used to obtain cellulosic wet sheet and membrane from wet sheet;

FIG. 2—Sequence in the purification and whitening of the cellulosic wet sheet (process conditions—time (Tₗ, volume (V₁) and number of times it is rinsed (Xₗ)—depend on the thickness and the size of the wet sheet formed);

FIG. 3—The cellulosic membrane surface viewed from above and magnified 38,000×by an electron microscope.

FIG. 4—Cellulosic membrane section viewed through an electron microscope, enlarged 6,500 times;

FIG. 5—Outline of the covered fermentation tray with temperature controlled by the circulation of liquids; this figure is composed of FIG. 5a—The tray without its covering seen from above, and 5b—transverse section of the tray (with the covering);

FIG. 6—Forming a semi-rigid end in the cellulosic wet sheet;

FIG. 7—Outline of the equipment used to drain and dry/dehydrate the wet sheets for the production of membranes;

FIG. 8—Detail A in FIG. 7;

FIG. 9—Detail B in FIG. 7;

FIG. 10—Detail C in FIG. 7;

FIG. 11—Detail D in FIG. 7;

FIG. 12—Detail of the coiling device;

FIG. 13—Outline of the upper cylinder lifting mechanism; and

FIG. 14—Comparative graph of the drying process.

### DETAILED DESCRIPTION OF THE INVENTION

This description is based on a preferred setting and makes reference to the figures attached. For clarification purposes alone, it will be divided into the following sections: culture medium, devices used, process, drying equipment used for the process, product obtained, use of the product, and tests.

Culture Medium

The culture medium required consists of the following ingredients and proportions:
filtered water—sand and activated charcoal filtering system;
0.2 to 12% in glucose mass;
0.1 to 7% of yeast extract
0.5 to 5% of ethanol

Covered Fermentation Tray

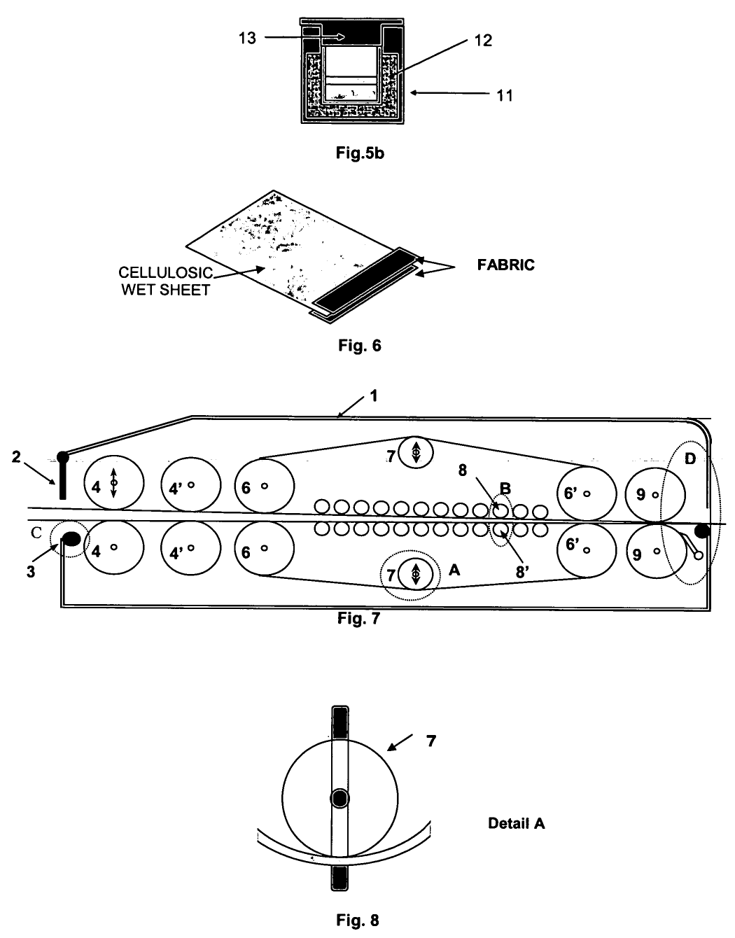

The tray used for fermentation consists of a tray (11) with double walls (12) and a covering (13); it is made of non-adherent material, preferably of fiberglass with a reinforced structure. The tray walls are 2 to 3 mm thick and form a duct where water circulates in order to keep the temperature at the ideal point for fermentation (FIG. 5).

The tray covering (13) is made of the same material and is embedded in the set. It consists of modules bonded together by rubber, preferably nitrile, thus allowing the liquid medium to be loaded into the tray by raising only the first and smaller module, reducing the exposure of the medium to contamination.

Processes

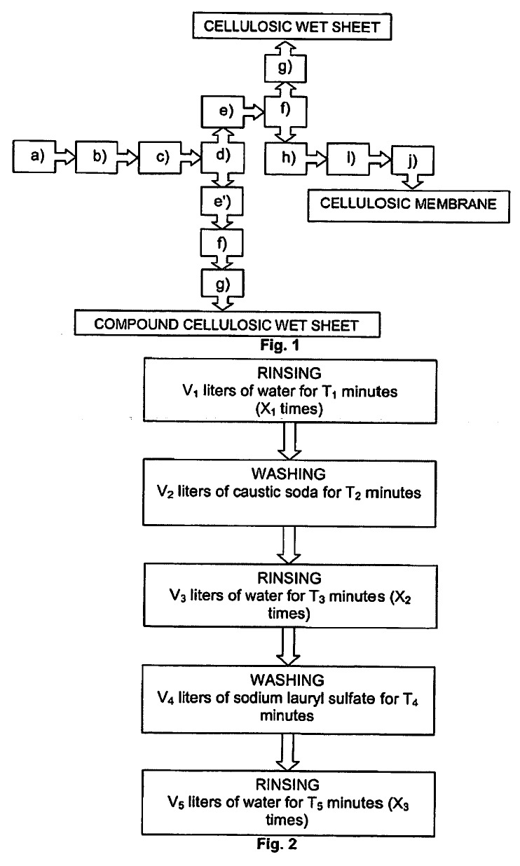

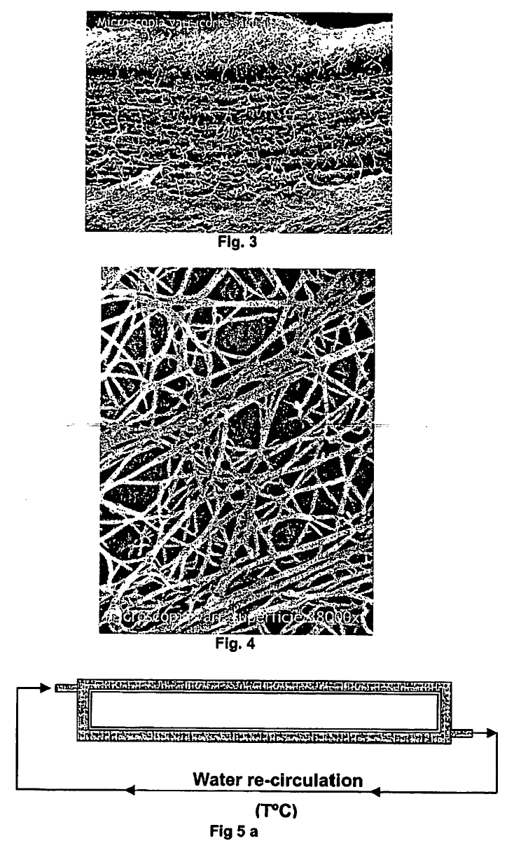

As noticed in FIG. 1, the process used to obtain cellulosic wet sheet, object of this invention, comprises the following steps:
a) Heating a solution containing 0.2 to 12% of glucose mass and 0.1 to 7% of yeast extract in water that was filtered through sand and activated charcoal, in a sanitary stainless steel mixer with steam heated jacket, at a temperature of 125° C. for 15 minutes, for sterilization purposes;
b) Cooling the solution till it reaches a temperature between 5 and 30° C.;
c) Adding 0.5 to 5% of ethanol and of 2 to 50% of the inoculum Acetobacter xylinum, followed by agitation of the solution until it is homogenized;
d) Transferring the solution to covered fermentation trays (FIG. 5) where it must rest for a period of 16 to 240 hours, at a temperature between 5 and 30° C.;
e) Collecting the cellulose wet sheets that are thus formed, varying from 0.25 to 200 mm in thickness;
e′) Adding screens or other artifacts of diverse materials to the surface of the wet sheet already pre-formed; it rests for another period of time that may vary from 16 to 240 hours at a temperature between 5 and 30° C.; the compound cellulose wet sheets thus formed with thickness varying from 0.25 to 40 mm are collected;
f) Forwarding the wet sheets to the whirlpool tank, where they are purified and whitened, according to the following sequence: rinsing, washing with 1 to 5% sodium hydroxide, rinsing, washing with 1 to 5% sodium lauryl sulfate and final rinsing (FIG. 2);
g) Packaging the wet sheets for shipping.

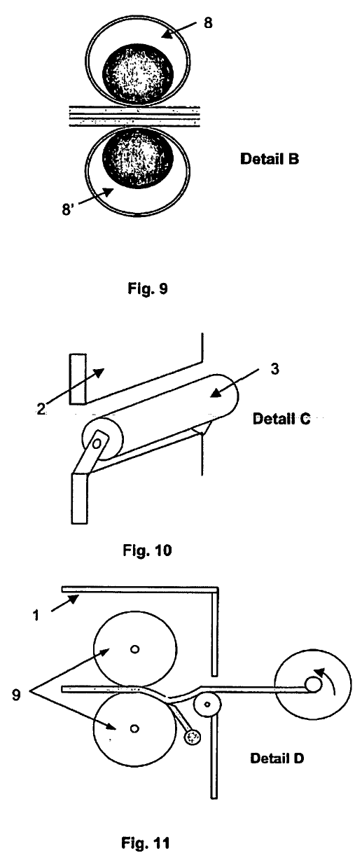

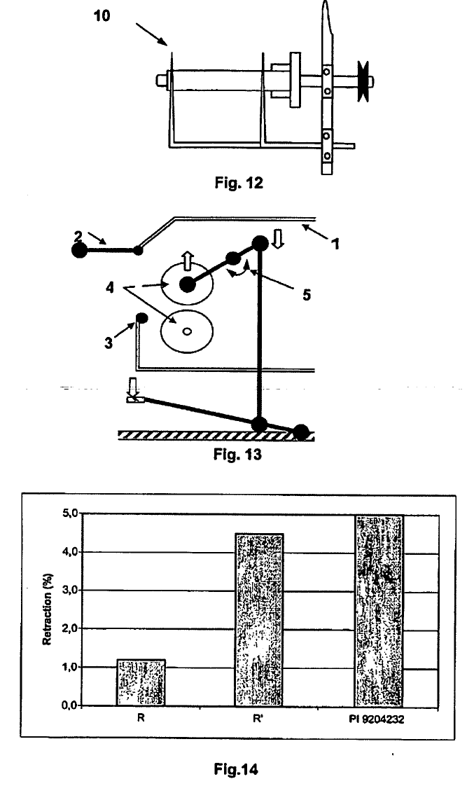

Optionally the wet sheets obtained through the process described above, with a level of humidity of 95 to 99.8%, will undergo the following process of draining and drying, according to the outline presented in FIG. 7:
h) In one of the extremities of the wet sheet two rectangles of an absorbent material are applied by pressure (one to each side of the wet sheet), so as to obtain a semi-rigid end that will not adhere to the drying material during this process (FIG. 6).
i) This extremity is inserted in the drying equipment through an idling roller (FIG. 10) and introduced between two pairs of draining cylinders, and from there to a pair of conveyor belts, being pressed between these belts with increasing force (from 0.5 to 8 kgf/cm2) that is applied by a series of small rollers heated by the hot water that circulates in their axles (FIG. 9); from there it goes to a pair of finishing cylinders, which may or may not be heated, so as to ensure a smooth surface for the membrane;
j) The membrane formed by the drying of the wet sheet is forwarded to a coiling device, where the product is coiled and stands ready for sterilization and/or shipping (FIGS. 11 and 12).

Treatment of Effluents

All effluents are recovered and treated before they are reused or disposed of, aiming at reducing water consumption, recovering thermal energy, and protecting the environment

Drying Equipment

The drying equipment is shown in FIGS. 7, 8, 9, 10 and 11, and consists of
Black steel plate (1) structure of appropriate thickness, forming a box with a lid (2) that is normally dosed and, for the purpose of ensuring the operator's safety, is provided with a power switch that arrests the motor when the lid is not totally shut, and an idling roller (3) coated with an absorbent material.
Two pairs (4 and 4′) of draining cylinders made of stainless steel coated with an absorbent material, 20 cm in diameter and 30 cm in width, where the upper cylinder (4) of the first pair is provided with a foot-actuated lifting mechanism (5, FIG. 13) and a lever system designed to permit the initial positioning of the wet sheet between the two cylinders;
Two pairs (6 and 6′) of stainless steel driving cylinders, 20 cm in diameter and 30 cm in width, with roller bearings, where the cylinders numbered (6) are not powered and the cylinders numbered (6′) are powered with a speed varying from 15 to 60 RPM;
Two felt (or any other water absorbing material) continuous conveyor belts measuring 2 m by 30 cm that are moved by the cylinders (6′); as well as two rollers (7) to control the tension on the belts, also made of stainless steel, with 10 cm in diameter;
Twelve or more pairs of stainless steel rollers (8, 8′), their external diameters measuring 5 cm each, the bottom ones (8′) heated by the passing of hot water or steam, through their axles. Each pair of rollers (8, 8′) makes it possible to apply a growing pressure to the conveyor belts made of absorbent material;
A pair of finishing cylinders (9) made of polished stainless steel, 20 cm in diameter and 30 cm long, each exerting pressure on the other, that can be heated by inner circulation of steam or hot water; and
A coiling device (10) made of carbon steel, as seen in detail in FIG. 12.

In order to ensure synchronism in the operation and control over the tension on the membrane so as to prevent it from being torn, every part of the equipment is put in motion through small sized gears and chains, as those used in bicycles.

Cellulose Wet Sheets

The cellulose wet sheets produced are made of randomly arranged pure cellulose micro-fibrils; they have a great capacity to retain liquids, keeping the humidity retained in its structure in excess of 90%; they resemble a whitish cartilage; they can resist high temperatures and may be submitted to autoclave treatment in liquid medium under high temperature and pressure without suffering any physical changes, being insoluble in organic solvents. Since they are processed in blenders, mills or equivalent, they result in a thick mass with a great power to retain liquids, fit for use as thickening and/or stabilizer in the food and beverage industry. In addition to these applications, the thick mass can be used in the production of lightweight plates extremely resistant to impact and bullet perforations. These plates are obtained by grinding the mass of cellulose micro-fibers in a liquid medium, then dehydrating the mass in such a way as to recast the material to form a plate to be molded as desired as to form and thickness. To cast a plate with approximately 3.5 cm, one would need a column of mass 45 cm high.

The cellulose wet sheets, when chemically cleaned and dehydrated, give origin to cellulose microfibrilar membranes that are impermeable to liquids and permeable to gases and are fit for use as a temporary replacement for the skin, propitiating the formation of a microenvironment that is ideal for tissue regeneration in living organisms that were badly wounded with loss of substance. Such membrane is characterized for being composed of pure cellulose microfibrils produced by microorganisms, randomly arranged, with an ideal weigh somewhere between 10 and 45 g/m2. This material is inert and biocompatible, and it does not cause allergic or rejection responses when applied over wounds in live organisms.

Product Applications

In this invention, the following applications for the bacterial cellulose wet sheets obtained in the process of this invention are proposed:
Cut in small pieces and grinded, they can be used as thickener or stabilizer in nonfat milk, juices and food in general;
In the making of artifacts obtained by drying and shaping the wet sheets (compound and not compound alike) in the forms desired for the final product, either pressed or non-pressed;
Cut in small cubes or strips and cooked in sugar syrup, they are a non-cholesterol fibrous food with the consistency of fruit or cooked squid, that can be used to compose canned food such as fruit salads and sweets, and for the manufacturing of candies that are similar to algae caramels or jelly beans.
Dried by the process that is part of this invention, they can be used to obtain extremely thin cellulosic membranes with characteristic properties such as permeability to gases and impermeability to liquids, for different uses as described below. The following applications are proposed in this invention for the bacterial cellulosic membranes obtained by the process in question:
As a device to obtain an environment suitable for “in vivo” culture in the regeneration of tissues in living organisms, both externally and internally, due to their biocompatibility that prevents rejection;
In Medicine, as a temporary substitute for the skin, mainly in dermal ulcers (diabetes, venous), bums, recovery of autograft-donor sites, membrane for ophthalmic use, odontological use, dressings for home use, professional use, veterinary use; as functional material in the making of hospital attire and disposable packaging for medical and nursing instruments whenever a microbial barrier is recommended, and, last but least, as thickener to replace sugar, starch, carboxymethylcellulose, and microcrystalline cellulose in the making of medicine tablets;
In the areas of Engineering and Safety, as a bulletproof material, upon treatment as described in the example.

Other uses for the Products Obtained

Submitted to a process of flash drying and vacuum or lyophilization, the wet sheets are transformed in a super absorbent and biocompatible material, and may be used as hemostatic internally and externally alike, in hospitals and surgery.

The membranes can also be used as diaphragm in the production of speakers and earphones.

### TEST EXAMPLE 1

### Cold Drying by Absorption

An assay for the purpose of observing the drying of a cellulosic wet sheet of bacterial origin was performed.

Experimental Procedure

The assay procedure consisted of the following steps:
The wet sheets, already purified and whitened, were cut in rectangles of approximately 4.0 cm×5.0 cm;
The thickness of the sample was measured between two rigid plastic plates with a pachometer, the length and width of the sample were also measured. Each dimension was measured three times so as to obtain an average value;
The sample was placed between two dry pieces of an absorbent fabric measuring 6.0 cm×8.0 cm;
This sandwich was placed between the two rigid plastic plates and the whole set was locked in place by fasteners that exerted a certain pressure;
After 3 minutes, the set was unfastened;
The four items above were repeated until there were no significant changes in the measurements obtained.
   Experimental Observation

During the first pressing of the wet sheet against the absorbent material, it was noticed that water was liberated from the set There was no further water leakage during the other pressings. It was also noticed an increase in the size of the pellicle. The thicker the pellicle, the greater the increase in size.

When the set was unfastened, it was noticed that the pellicle adhered to the surface of the absorbent material, and had to be loosed with the help of an external device.

In order to measure the pellicle, it was put on a glass surface. Adherence to this surface was even greater. While detaching the pellicle from the surface, it was noticed that it had become slightly damaged (wrinkled).

In the last steps of the drying process, when the pellicle edges were almost dry, measuring was hindered because the pellicle would not adhere to the glass surface and it was impossible to keep it stretched (it became slightly crumpled).

Data Treatment

1. Calculating the Pellicle Retraction After Drying

When the drying process proposed in this assay was completed, the pellicle retraction was measured. The retraction R (in %) was calculated as:
R=−[(D−Di)/Di]100 (1)
where:
D is the final width or length average measurement
Di is the Initial width or length average measurement

It was also noticed an increase in the size of the wet sheet after the first drying cycle. Therefore, the retraction of the wet sheet was calculated using the maximum measurement obtained (R′in %):
R′=−[(D max−Di)/Di]100 (2)
where:
Dmax is the maximum average measurement;

The results obtained were:

TABLE 1
Wet sheet retraction when dried by the method proposed herein

| | | | |
|---|---|---|---|
| Length | Width | Length | Width |

| | | |
|---|---|---|
| Sample | R (%) | R′(%) |

| | | | | |
|---|---|---|---|---|
| 1 | 3.63 | 5.44 | 4.39 | 5.86 |
| 2 | −2.72 | −2.43 | 4.09 | 4.40 |
| 3 | −0.20 | 3.36 | 4.37 | 3.52 |
| Average | 0.23 | 2.12 | 4.28 | 4.59 |

| | |
|---|---|
| 1.2 | 4.5 |

2. Calculating Expansion and Reduction in Thickness During First Pressing

As described above (see Experimental Observation), after the first pressing the membrane expanded and its thickness was reduced. Expansion and reduction figures are shown below:

TABLE 2
Expansion of the wet sheet after first pressing

Expansion (%)

| Sample | Length | Width |
|---|---|---|
| 1 | 0.65 | 0.44 |
| 2 | 7.11 | 7.15 |
| 3 | 4.78 | 0.16 |
| Average | 4.2 | 2.6 |

3.4

TABLE 3
Reduction of the wet sheet thickness after first pressing

Thickness (10⁻³ cm)

| Sample | Initial | 2^{nd} measurement | Thickness reduction (%) |
|---|---|---|---|
| 1 | 225 | 28 | 87.6 |
| 2 | 386 | 46 | 88.1 |
| 3 | 337 | 85 | 74.8 |

Conclusions

It was concluded from the assay that after a certain number of drying cycles the cellulosic wet sheet is dry to touch. The experiment did not measure the humidity of material or its balanced humidity. The sample thickness and its mass reduction weren't measured as well.

It was noticed that the retraction in the dimensions of the membrane was of the order of 4.5%. From the values obtained, it is also perceived that the larger the reduction in the sample's initial thickness, the larger the expansion of its dimensions.

The retraction figures presented in patent PI 9204232 are of the same order of magnitude (5%) if compared to the maximum value of the dimension measured, R′(wet sheet after first pressing). If the comparison is made considering the original dimensions of the wet sheet, where the average retraction obtained was of the order of 1.2% (R), the figures reported in above mentioned patent are quite unfavorable. This data can be better observed in FIG. 14.

It is also worth noticing that in the method described in the patent mentioned above there is an additional cost related to energy and labor required in freezing and thawing of the membranes before the drying procedures, and that in our case there are no additional operations involved.

### TEST EXAMPLE 2

### Calender Drying

An assay for the purpose of observing the drying of a cellulosic wet sheet of bacterial origin by pressing it against absorbent fabric while passing through two metal cylinders

Experimental Procedure

The assay procedure consisted of the following steps:
Place the sample between two pieces of absorbent fabric
Press the set fabric/wet sheet/fabric through the metal cylinders 10 times;
Exchange one of the sides of the fabric and press the set through the metal cylinders another 10 times. Keep exchanging one side of the fabric (always the same), pressing through the cylinders, and observing until the material is dry.
Observe the reduction in thickness and if there is adherence of the membrane to the roller.
   Experimental Observations

It was noticed that there was a great amount of water drained during the first times the sample was pressed through the rollers. When the fabric was separated from the set in order to switch one of the sides, it was noticed that the membrane formed had adhered to the fabric and it was necessary a mechanical effort to remove it.

After the fourth time the fabric was exchanged, it was noticed that the membrane was almost dry. It was then separated from the absorbent fabric, and in a few seconds it had dried in the air.

The initial and final lengths of the sample were measured as shown in Table 1.

TABLE 1
Measurements of the sample before and
after the draining and drying process

| | Length | Width |
|---|---|---|
| Before | 4125.10⁻³ cm | 1457.10⁻³ cm |
| After | 4380.10⁻³ cm | 1552.10⁻³ cm |
| Variation (%) | +6% | +6% |

Conclusion

Draining of the cellulosic wet sheet by pressing it against an absorbent fabric while passing through two stainless steel cylinders is perfectly possible and viable.

Also, it was observed an increase in the membrane dimensions as compared to the initial measurements of the wet sheet. Measurements were not taken throughout the assay, so it is not possible to determine if there was a retraction of the pellicle with reference to a possible maximum value.

### TEST EXAMPLE 3

### Drying by Absorption and Heat

An assay for the purpose of observing the drying of a cellulosic wet sheet of bacterial origin by pressing it against absorbent fabric while passing through two metal cylinders, with the use of heat

Experimental Procedure

The assay procedure consisted of the following steps:
Place the sample between two pieces of absorbent fabric;
Press the set fabric/wet sheet/fabric through the metal cylinders 3 times;
Place the set between two heated metal surfaces, pressing for 1 minute;
Pass the ser through the cylinders 10 times;
Repeat the two previous operations until the set is totally dry.
   Experimental Observations

It was noticed that by repeating the procedure twice the membrane was dried.

Another version of the assay was performed: the sample was not initially drained by passing through the metal cylinders. The set fabric/membrane/fabric was placed between two metal surfaces heated to a higher temperature than in the previous assay, and pressure was exerted on it. Every 30 seconds the state of the material was observed (by lifting the upper surface). After 10 minutes, the sample was dry.

In this assay, the sample was extremely bonded to the absorbent fabric. In trying to remove it, its fibers were ruptured for part of it had adhered to the absorbent fabric.

Conclusion

Assays where heat was used led to the conclusion that an initial draining step speeds the drying process, decreasing the time of exposure to heat, resulting in lower costs.

Another conclusion is that there should be a greater control over the heating temperature, for if the temperature is too high there might be a rupture of the membrane and adherence to the absorbing fabric.

Final Considerations

In as much as this invention refers to a production in industrial scale, avoids the need for humidity control, avoids the need for forced air circulation, doesn't form lamella in the zooglea thus preventing a waste of material and labor to pull it off, and avoids the need to stretch the wet sheets on stenters to dry or its freezing, it is more economical and yields better results; because it uses selected recycled or non-recycled inoculums, a culture medium with components of controlled quality and standardized procedures, it can assure consistent quality for the bacterial cellulose wet sheet and the cellulosic membrane.

There are no records of the production in large scale of bacterial cellulose wet sheets, using inoculums that are reproduced in an environment that is separated from the production area in order to prevent contamination, using quality controlled sterilized industrial culture medium, with fermentation in invented covered trays featured with an autonomous system of temperature control, avoiding the need for clean air and humidity control, purification and whitening system by whirling, semi-continuous dehydration/drying in invented equipment, membrane coiling, effluents reusing and treatment, rendering the process efficient, economical, and clean.

The benefits brought by the use of these products, first in the Medical area, with immediate pain relief, non-replacement of the dressing (thus avoiding that the protection formed reopens and gets torn), shorter convalescence period, shorter hospital stay, and also in the dietary area (products made of pure cellulose fibers) and in the security area (lightweight bulletproof plates) provide this application for a patent with value to the human race as whole.

1. “Process used to obtain cellulosic wet sheet” characterized by the following steps:
a) heating a solution containing 0.2 to 12% of glucose mass and 0.1 to 7% of yeast extract in water that was filtered through sand and activated charcoal, in a sanitary stainless steel mixer with a steam heated jacket, at a temperature of 125° C. for 15 minutes, for sterilization purposes;
b) cooling the solution till it reaches a temperature between 5 and 30° C.;
c) adding 0.5 to 5% of ethanol and of 2 to 50% of the inoculum Acetobacter xylinum, followed by agitation of the solution until it is homogenized;
d) transferring the solution to covered fermentation trays where it must rest for 16 to 240 hours, at a temperature between 5 and 30° C.;
e) collecting the cellulose wet sheets that are thus formed, varying from 0.25 to 200 mm in thickness;
f) forwarding the wet sheets to the whirlpool tank, where they are purified and whitened, according to the following sequence: rinsing, washing with sodium hydroxide 1 to 5%, rinsing, washing with 1 to 5% sodium lauryl sulfate and final rinsing;
g). packaging the wet sheets for shipping.

2. “Process used to obtain cellulosic membrane” from the wet sheet as described in claim 1, characterized by the following steps:
h) in one of the extremities of the wet sheet, two rectangles of an absorbent material are applied by pressure (one to each side of the wet sheet), so as to obtain a semi-rigid end that will not adhere to the drying material during this process;
i) this extremity is inserted in the drying equipment through an idling roller and introduced between two pairs of draining cylinders, and from there to a pair of conveyor belts, being pressed between these belts with increasing force (from 0.5 to 8 kgf/cm2) that is applied by a series of small rollers heated by the hot water that circulates in their axles; from there it goes to a pair of finishing cylinders, which may or may not be heated, so as to ensure a smooth surface for the membrane;
j) the membrane formed by the drying of the wet sheet is forwarded to a coiling device, where the product is coiled and stands ready for sterilization and/or shipping.

3. Process used to obtain cellulosic wet sheet according to claim 1, characterized by optionally comprising a step where screens or other artifacts of diverse materials are added to the surface of the wet sheet already pre-formed; then this set rests for another period of time that may vary from 16 to 240 hours at a temperature between 5 and 30° C. followed by the collecting of the compound cellulose wet sheets thus formed varying from 0.25 to 40 mm in thickness;

4. “Culture medium” used in the process in claim 1, characterized by containing water filtered; 0.2 to 12% of glucose mass; 0.1 to 7% of yeast extract, and 0.5 to 5% of ethanol.

5. “Fermentation tray” used in the process in claim 1, characterized by its consisting of a tray (11) with a double wall (12) and a covering (13); made of non-adherent material, preferably fiberglass with a reinforced structure. Said tray walls are 2 to 3 mm thick and form a duct where water circulates in order to keep the temperature at the ideal point for fermentation.

6. Fermentation tray according to claim 5, characterized by the fact that the tray covering (13) is made of the same material of the tray (11) and is embedded in the set, said tray covering consists of modules bonded together by rubber, preferably nitrile, thus allowing the liquid medium to be loaded into the tray by raising only the first and smaller module, reducing the exposure of the medium to contamination.

7. Equipment used to obtain the membrane, according to the process in claim 2, characterized by its consisting of:
a black steel plate (1) structure of appropriate thickness, forming a box with a lid (2) that is normally closed and, for the purpose of ensuring the operator's safety, is provided with a power switch that arrests the motor when the lid is not totally shut, and an idling roller (3) coated with an absorbent material.
two pairs (4 and 4′) of draining cylinders made of stainless steel coated with an absorbent material, 20 cm in diameter and 30 cm in width, where the upper cylinder (4) of the first pair is provided with a foot-actuated lifting mechanism (5) and a lever system designed to permit the initial positioning of the wet sheet between the two cylinders;
two pairs (6 and 6′) of stainless steel driving cylinders, 20 cm in diameter and 30 cm in width, with roller bearings, where the cylinders numbered (6) are not powered and the cylinders numbered (6′) are powered with a speed varying from 15 to 60 RPM;
two felt (or any other water absorbing material) continuous conveyor belts measuring 2 m by 30 cm that are moved by the cylinders (6′); as well as two rollers (7) to control the tension on the belts, also made of stainless steel, with 10 cm in diameter;
twelve or more pairs of stainless steel rollers (8, 8′), their external diameters measuring 5 cm each, the bottom ones (8′) heated by the passing of hot water or vapor through their axles; each pair of rollers (8, 8′) makes it possible to apply increasing pressure to the conveyor belts made of absorbent material;
a pair of finishing cylinders (9) made of polished stainless steel, 20 cm in diameter and 30 cm long, each exerting pressure on the other, that can be heated by inner circulation of steam or hot water; and
a coiling device (10) made of carbon steel.

8. “Cellulosic membrane” characterized for being inert, biocompatible and composed of pure cellulose microfibrils produced by microorganisms, randomly arranged, with an ideal weigh somewhere between 10 and 45 g/m2, being said membrane permeable to gases and impermeable to liquids.

9. “Process used to obtain a wet sheet” in accordance with claim 1, characterized by the fact that all effluents are recovered and treated before they are reused or disposed of in the sewer system.

10. “Process used to obtain a wet sheet” in accordance with claim 1, characterized by the fact that the wet sheet obtained is made of randomly arranged pure cellulose microfibrils with a great capacity to retain liquids, keeping the humidity retained in its structure in excess of 90%, and that said wet sheet is cartilage-like and of a whitish color.

11. Process used to obtain a wet sheet in accordance with claim 1, characterized by the fact that the sheets obtained resist high temperatures and optionally may be submitted to autoclave treatment in liquid medium under high temperature and pressure without suffering any physical changes;

12. Process used to obtain a wet sheet in accordance with claim 1, characterized by the fact that the sheets obtained are insoluble in organic solvents, and that they may be processed in blenders, mills or the equivalent, resulting in a thick mass with a great power to retain liquids.

13. Process used to obtain a wet sheet in accordance with claim 1, characterized by the fact that the sheets obtained are used as thickening and/or stabilizer in the food and beverage industry.

14. Process used to obtain a wet sheet in accordance with claim 1, characterized by the fact that the sheets obtained in the form of a thick mass can be used also in the production of lightweight plates extremely resistant to impact and bullet perforations.

15. Process used to obtain a wet sheet in accordance with claim 14, characterized by the fact that said plates are obtained by grinding the wet sheet in a liquid medium, then dehydrating the mass of cellulose microfibrils in such a way as to recast the material to form a plate to be molded as desired as to form and thickness.

16. Process used to obtain a wet sheet in accordance with claim 1, characterized by the fact that the sheets obtained are used as thickeners or stabilizers in nonfat milk, juices and food in general; used in the making of artifacts obtained by drying and shaping the wet sheets (compound and not compound alike) in the forms desired for the final product, either pressed or non-pressed; included in the composition of canned foods; used in the production of sweets; and used to obtain extremely thin cellulosic membranes with characteristic properties such as permeability to gases and impermeability to liquids.

17. Process used to obtain a wet sheet in accordance with claim 1. characterized by the fact that the sheets obtained, submitted to a fast vacuum drying process or to lyophilization, are transformed in a super absorbent and biocompatible material, a hemostatic both of internal and external use in hospital and surgical applications.

18. Process used to obtain a membrane in accordance with claim 2, characterized by the fact that said membrane is composed of pure cellulose microfibrils produced by microorganisms, randomly arranged, with an ideal weigh somewhere between 10 and 45 g/m2.

19. Process used to obtain a membrane in accordance with claim 2, characterized by the fact that said membrane is inert and biocompatible, so as not to cause allergic or rejection responses when applied over wounds in live organisms.

20. Process used to obtain a membrane in accordance with claim 2, characterized by the fact that said membrane's most distinguished feature is its permeability to gases and impermeability to liquids.

21. Process used to obtain a membrane in accordance with claim 2, characterized by the fact that the membranes obtained are used as a device to obtain an environment suitable for “in vivo” culture in the regeneration of tissues in living organisms, both externally and internally, due to their biocompatibility that prevents rejection; as a temporary substitute for the skin, mainly in dermal ulcers, burns, recovery of autograft-donor sites, membrane for ophthalmic use, odontological use, dressings for home use, professional use, veterinary use; as functional material in the making of hospital attire and disposable packaging for medical and nursing instruments whenever a microbial barrier is recommended, and also as thickener to replace sugar, starch, carboxymethylcellulose, and microcrystalline cellulose in the making of medicine tablets; as engineering and safety material, specially as a bulletproof material; and as diaphragm in the production of speakers and earphones.
